(19) United States
(12) Patent Application Publication
(10) Pub. No.: US 2015/0351944 A1
(43) Pub. Date: Dec. 10, 2015

(54) GASTRIC IMPLANT AND METHOD FOR USE OF SAME

(71) Applicant: Donald J. Waldrep, Thousand Oaks, CA (US)

(72) Inventor: Donald J. Waldrep, Thousand Oaks, CA (US)

(21) Appl. No.: 14/828,265

(22) Filed: Aug. 17, 2015

Related U.S. Application Data
(63) Continuation of application No. 13/088,319, filed on Apr. 15, 2011, now abandoned.
(60) Provisional application No. 61/324,456, filed on Apr. 15, 2010.

(51) Int. Cl. A61F 5/00

(52) U.S. Cl. CPC A61F 5/0083; A61F 5/0026

(57) ABSTRACT

An implant device and method for inducing weight loss is provided wherein the implant device deforms the stomach wall thereby reducing the effective stomach volume without connection or access to the inner stomach. The implant device has a shape to provide for deformation of the stomach without retention within the inner stomach.

### CROSS-REFERENCE TO RELATED APPLICATION(S)

The present application is a continuation of U.S. patent application Ser. No. 13/088,319, filed Apr. 15, 2011, which claims priority to and the benefit of U.S. Provisional Application Ser. No. 61/324,456, filed on Apr. 15, 2010, the entire contents of which are incorporated herein by reference.

### FIELD

The present invention relates to a gastric (abdominal) implant for inducing weight loss in a patient by reducing the effective volume of the patient's stomach.

### BACKGROUND

Presently available gastric implants and treatments for inducing weight loss require intra-gastric procedures and implantation. Specifically, some of the presently available gastric implants require a connection to a port-type device for adjustment. For some implants, attachment parts are placed on both sides of, or even traversing, the gastric wall. Breaching of the enteric lumen (inner digestive tract, inner stomach) by perforation or chronic application of pressure increases the risk of infection and complications. Presently available gastric implants require compromising the integrity of the gastric wall. Gastric balloons require maintenance and routine interventional techniques e.g. inflation, and are inherently tenuous in that deflation cannot be wholly prevented.

For example, US 2005/0261712 to Balbierz et al. discloses a gastric implant that requires a retention band positioned intra-gastrically or trans-gastrically to anchor the implant to the outer gastric wall. This intra-gastric anchor presents a significant likelihood of erosion, perforation, and implant migration. Accordingly, there is a need for a low-maintenance extra-gastric implant that can be surgically implanted using minimally invasive procedures, as well as traditional techniques. An implant device that is exclusively extra-gastric does not require breaching the integrity of the gastric wall, and does not impart significant risks associated with erosion or malposition of multiple parts of existing or proposed gastrically related implant devices.

### SUMMARY

A gastric implant is provided preferably requiring only extra-gastric, minimally invasive surgery. In one embodiment of the present invention, a gastric implant device is provided for decreasing the effective volume of a patient's stomach, wherein the implant device includes a space occupying member made from biocompatible material adapted to be positioned at an outer stomach wall and to deform the stomach by forming two ends of the outer stomach wall without breaching the inner stomach cavity, and a means for enclosing the space-occupying member by connecting the two ends of the outer stomach wall without breaching the inner stomach cavity.

In a second embodiment of the present invention, the space-occupying member is expandable.

In a third embodiment of the present invention, the means for enclosing the space-occupying member is a seromuscular means selected from the group consisting of sutures, wire devices, mechanical pressure devices, stapling devices, shape-memory metals, and combinations thereof.

In a fourth embodiment of the present invention, the space-occupying member is made from biocompatible material selected from the group consisting of foam, silicone, silastic, polytetrafluoroethylene (PTFE), polyethylene, polypropylene, polyglactin, Seprafilm®, Interceed®, REPEL-CV®, ADEPT®, Biodesign® Surgisis®, human acellular tissue, porcine collagen, bovine pericardium, textured synthetics, and combinations thereof.

In a fifth embodiment of the present invention, the space-occupying member has a wand attached thereto.

In a sixth embodiment of the present invention, the wand is hollow and may contain wires having electrode leads.

In a seventh embodiment of the present invention, the implant device has an extension attached thereto for encircling the stomach.

In an eighth embodiment of the present invention, the space-occupying member has a first pole and a second pole, a spine, and an arch, wherein the spine and the arch are positioned opposite each other between the first pole and the second pole.

In a ninth embodiment of the present invention, the space-occupying member has a first arch and a second arch with a groove positioned therebetween, wherein the first and the second arches and the groove are positioned opposite the spine.

In a tenth embodiment of the present invention, the space-occupying member has a length from the first pole to the second pole that is of from about 5 cm to about 25 cm, and the circumference of the space-occupying member at the midpoint between the first pole and the second pole is of from about 1 cm to about 15 cm.

In an eleventh embodiment of the present invention, a method for decreasing the effective volume of a patient's stomach without accessing the inner stomach cavity is disclosed, including the steps of providing a space-occupying member to the outer stomach wall of a patient; deforming the stomach with the space-occupying member to form two ends; encasing the space-occupying member in the deformed stomach; and approximating the two ends of the outer stomach wall together to thereby enclose the space-occupying member in the outer stomach wall of the deformed stomach.

In a twelfth embodiment of the present invention, the method for decreasing the effective volume of a patient's stomach includes removing the space-occupying member prior to approximating the two ends together.

In a thirteenth embodiment of the present invention, the method for decreasing the effective volume of a patient's stomach includes a wand attached to the space-occupying member and the deforming of the stomach includes positioning the space-occupying member using the wand.

In a fourteenth embodiment of the present invention, the method for decreasing the effective volume of a patient's stomach includes detaching the wand from the space-occupying member after approximating the two ends.

In a fifteenth embodiment of the present invention, the method for decreasing the effective volume of a patient's stomach includes a hollow wand that contains wires having electrode leads attached directly to the space-occupying member, and detaching the wand from the space-occupying member, wherein the wires having electrode leads remain attached to the space-occupying member.

In a sixteenth embodiment of the present invention, the electrical leads are used for applying electrical stimulation to the outer stomach wall.

In a seventeenth embodiment of the present invention, the method for decreasing the effective volume of a patient's stomach loss includes encircling the stomach with an extension wrap.

In an eighteenth embodiment of the present invention, the approximating of the two ends is carried out by a seromuscular means selected from the group consisting of sutures, wire devices, mechanical pressure devices, stapling devices, shape-memory metals, and combinations thereof.

In a nineteenth embodiment of the present invention, a kit is provided that includes at least a first implant device and a second implant device, each of the implant devices includes a space-occupying member made from biocompatible material adapted to be positioned at an outer stomach wall and to deform the stomach by forming two ends of the outer stomach wall without breaching the inner stomach cavity, wherein each of the implant devices is different in size from another implant device of the kit.

In a twentieth embodiment, the space-occupying member of each of the implant devices of the kit further includes a first pole and a second pole; an arch; and a spine, wherein the arch and spine are positioned opposite each other in between the first pole and the second pole, wherein the set of dimensions comprises a length from the first pole to the second pole that is of from about 5 cm to about 25 cm, and a circumference of the space-occupying member at the midpoint between the first pole and the second pole is of from about 1 cm to about 15 cm.

In a twenty-first embodiment of the present invention, the space-occupying member of at least one of the implant devices of the kit further comprises a first arch and a second arch with a groove positioned therebetween, wherein the first and second arches and the groove are positioned opposite the spine.

In a twenty-second embodiment, the space-occupying member includes a computer or processor.

### BRIEF DESCRIPTION OF THE DRAWINGS

FIG. 1 is a schematic of a stomach and an implant device according to the present invention;

FIG. 2 is a schematic of a stomach being implanted with an implant device according to an embodiment of the present invention;

FIGS. 3A-3C are schematics of an implant device according to embodiments of the present invention;

FIG. 4 is a schematic of a stomach being implanted with an implant device according to embodiments of the present invention;

FIG. 5. is a schematic of an imbricated stomach with a seromuscular closure of the approximated ends of the outer stomach wall, according to embodiments of the present invention;

FIG. 6 is a schematic of a stomach with implant device therein after approximation/closure, according to embodiments of the present invention;

FIG. 7 is a schematic of a stomach implanted with an implant device and a wand according to an embodiment of the present invention;

FIG. 8 is a schematic of a stomach implanted with an implant device, a wand, electrode leads, electrical wires and a power source according to an embodiment of the present invention;

FIG. 9 is a schematic of a stomach implanted with an implant device, a wand, electrode leads, electrical wires and a power source according to an embodiment of the present invention; and

FIGS. 10A-10B; FIG. 10A is a schematic of an extension wrap around a stomach, and FIG. 10B is a top-view schematic of a stomach inverted (deformed) with an implant device and an extension wrap around the inverted stomach according to an embodiment of the present invention.

FIG. 11 is a schematic of the implant device showing the space-occupying member with a computer or processor.

### DETAILED DESCRIPTION

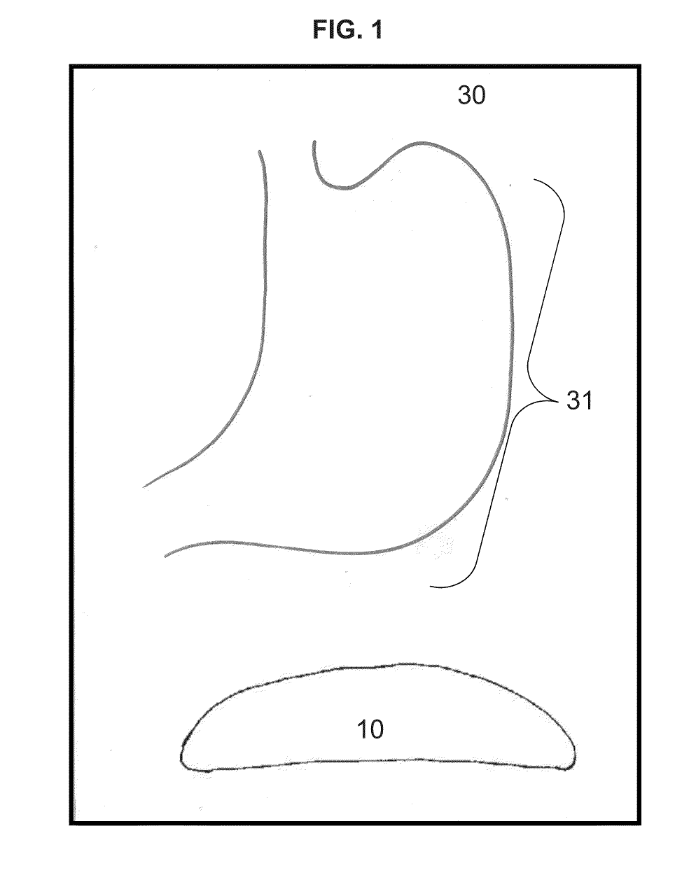

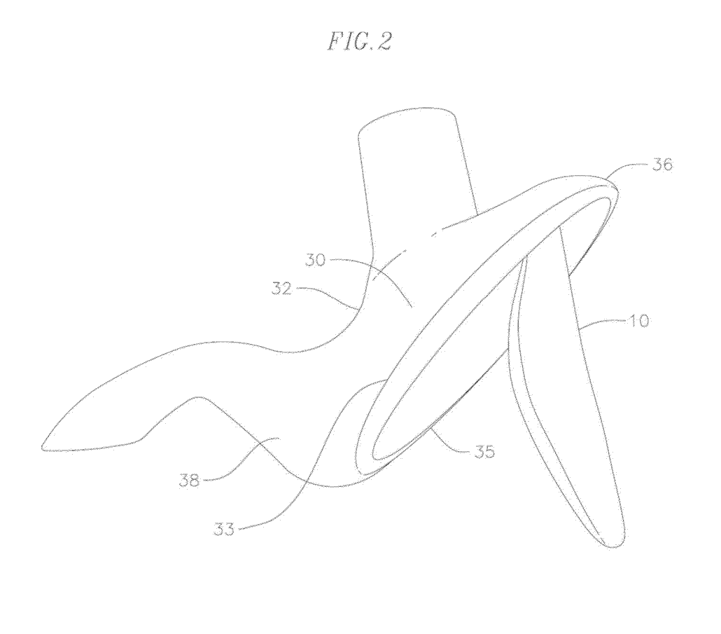

An implant device that is preferably implanted using minimally invasive techniques, as well as traditional techniques, is disclosed herein. The implant device has a space-occupying member (10) that is made of a biocompatible material disposed to be surrounded by, and outside of, the stomach (30) (FIG. 1). The space-occupying member of the implant device creates a sustained geometric deformation of the stomach by inversion of the “greater curve” (i.e. left, lateral outer wall) (31) of the stomach resulting in a significant decrease in the intra-luminal stomach volume and its compliance. In one embodiment, the sustained geometric deformation of the stomach by inversion of the greater curve by the space-occupying member of the implant device, results in a significant decrease in the intra-luminal stomach volume and its compliance. As shown in FIG. 2, the greater curve (31) of the stomach (30) is inverted along its length from fundus (36) to antrum (38). By physically limiting the volume of food that can be held by the native stomach, weight loss can occur.

In FIG. 2, the space-occupying member of the implant device (10) according to one embodiment of the present invention, is implanted onto the outer wall of the stomach such that the stomach encases the space-occupying member (10) and folds onto itself—i.e. imbrication of the stomach—creating an anterior gastric fold (33) and a posterior gastric fold (35). The folds (33, 35) are also referred to herein as “ends.” Once the space-occupying member of the implant device (10) is encased, the two ends (33, 35) of the stomach are approximated together (i.e., appositioned) around the implant.

In one embodiment, the space-occupying member (10) is made from any biocompatible material or any combination of materials that renders the implant device suitable for implantation and permanent placement within the human or animal body, and specifically encased within the two ends (33, 35) of the stomach.

In one embodiment, the biocompatible material is homogenous throughout the space-occupying member of the space-occupying member. Alternatively, the outermost material of the space-occupying member differs from the inner material(s). Possible biocompatible materials may include but are not limited to, at least one from the group including foam (e.g. quantum foam, polyurethane foam (foam rubber), polystyrene, and phenolic), silicone, silastic, polytetrafluoroethylene (PTFE), a mesh made from, e.g. polyethylene, polypropylene, polyglactin, Seprafilm®, Interceed®, REPEL-CV®, ADEPT®, Biodesign® Surgisis®, a biologic (e.g. dermal or connective tissue derived materials including human acellular tissue, porcine collagen, and bovine pericardium), textured synthetics (e.g. Gore® Dualmesh®), and combinations thereof.

The outermost material of the space-occupying member may be composed of an artificial material that is permanent or resorbing. The outermost material may be biologic and integrate to native tissue or resorb over time. In one embodiment, the outermost material is designed to promote or inhibit tissue formation (adhesions) either on the whole outer implant or on specific regions of the implant. Exemplary materials that promote tissue formation include Biodesign® Surgisis®, a biologic (e.g. dermal or connective tissue derived materials including human acellular tissue, porcine collagen, and bovine pericardium), textured synthetics (e.g. Gore® Dualmesh®), and synthetic materials including polyethylene, polypropylene, and polyglactin meshses. Exemplary materials that inhibit tissue formation include Seprafilm®, Interceed®, REPEL-CV®, and ADEPT®.

In one embodiment, the inner material of the space-occupying member may be comprised of foam with a non-foam outer layer. In one embodiment, the inner material is foam that expands. The foam may irreversibly expand. The outer material may also be foam.

In one embodiment, the space-occupying member made of biocompatible material is a solid structure having an outer material and inner material as described herein. The space-occupying member may also have an outer material with a porous inner material. Alternatively, the space-occupying member is hollow having an outer material and an inner space.

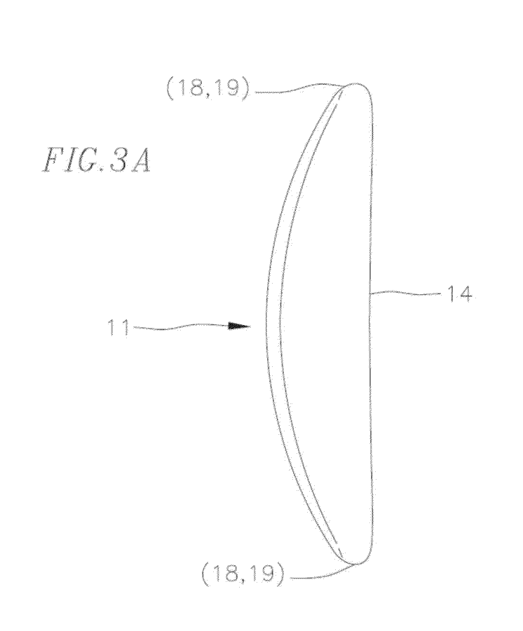

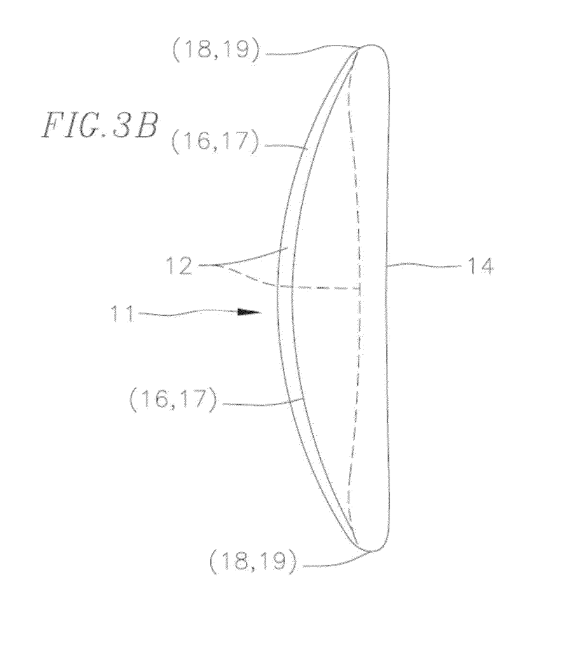

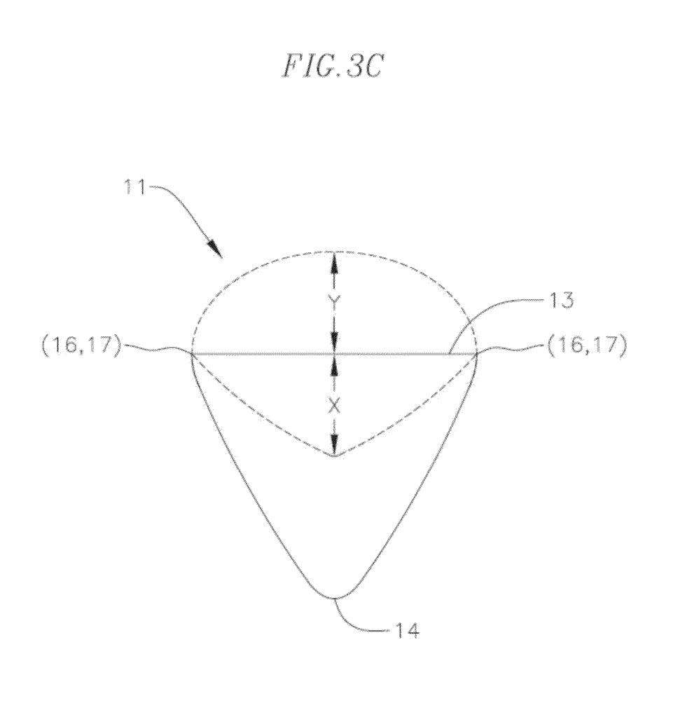

The space-occupying member (10) according to the present invention is shown in FIGS. 3A-3C. In one embodiment (FIG. 3A), the space-occupying member of the implant device has two poles (18, 19), a spine (14) and an arch (11), with the spine and arch positioned opposite each other (as shown, FIG. 3A) in between the poles (18, 19). The arch (11) may be convex having a height of y above the arch plane (13), as shown in FIG. 3C, wherein y is up to 5 cm. Alternatively, the “arch” (11) is flat, and therefore, does not necessarily resemble an arch when y is 0.

In one embodiment, the space-occupying member of the implant device has an arch (11) with a groove (12), both of which are opposite the spine (14) (FIG. 3B). As shown, the groove (12) renders two arches (16, 17). As shown in FIG. 3C, the groove (12) has a depth x, wherein x is up to 5 cm. In the context of providing the implant to the patient's stomach (FIG. 2), with respect to the fundus (36) and the antrum (38), as well as the posterior and anterior gastric ends (33, 35). one of the two arches (16, 17) is a posterior arch and the other is an anterior arch, and the two poles (18, 19) will have a “north” and “south” orientation.

As described and/or shown, the space-occupying member of the implant device of the present invention may have a groove (12) depressed in the arch (11), or a smooth convex arch (11), or a smooth flat “arch” along the position of the arch (11).

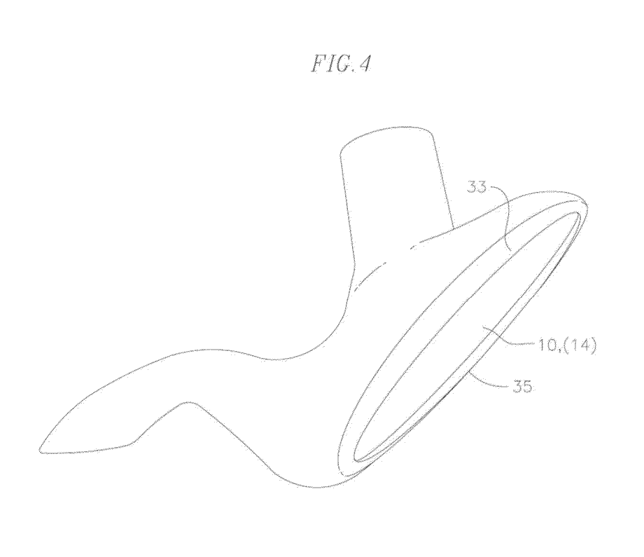

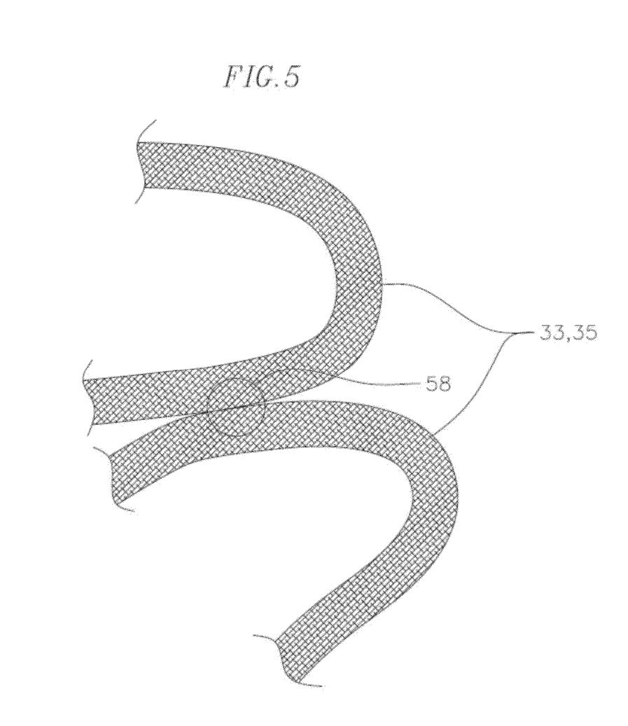

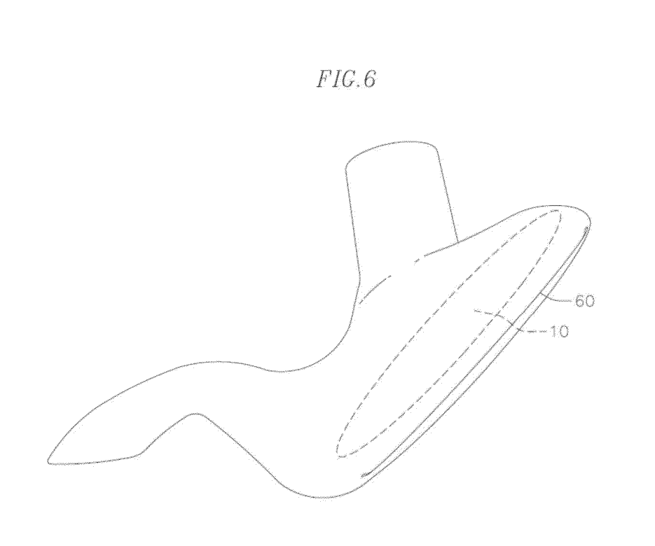

A space-occupying member as shown in FIGS. 3A and 3B, is positioned between the gastric ends (folds) (33, 35) with the arch (11) toward the stomach, such that after implantation, the spine (14) is the only part of the space-occupying member of the implant device exposed prior to approximation of the ends as shown in FIG. 4. Approximation of the ends is carried out using a seromuscular closure (58) (FIG. 5). Any known seromuscular closure means may be used to avoid breaching of the inner stomach cavity (i.e. to avoid transmural gastric penetration to the inside of the stomach). A seromuscular closure would puncture the serosa, muscularis and possibly the submucosa layers of the stomach wall, but would not go through the mucosa layer. Complete approximation of the ends to form a complete seromuscular closure (60) is shown in FIG. 6. For example, the means for enclosing the space-occupying member by seromuscular means includes, but is not limited to, sutures, wire devices, mechanical pressure devices, stapling devices, shape-memory metals, and combinations thereof.

In one embodiment, the space-occupying member of the implant device expands by vacuum release. For example, the space-occupying member is made of compressible material, e.g. foam, wherein when packaged under vacuum, the implant device is reduced in size. Prior to release of the vacuum, the packaged implant is surgically provided proximal to the outer stomach wall. Upon release of the vacuum, the implant expands and is then implanted into the outer stomach as described herein. Expansion of the space-occupying member upon vacuum release is advantageous for implantation using laparoscopic techniques wherein the space-occupying member of the implant device would be passed through a trocar site. A space-occupying member that expands upon vacuum release could also be used for natural orifice techniques (e.g. endoscopy), as well as traditional “open” techniques.

The space-occupying member of the present invention can be sized using controlled inflation, controlled expansion, and/or mechanical adjustment, by having preformed sizes and shapes, and combinations thereof. In one embodiment, the space-occupying member has a selected preset size, wherein the selected preset size is selected from a group of implants having a length between the two poles (18, 19) of about 5 cm to about 25 cm, and a mid-implant circumference of about 1 cm to about 15 cm. Mid-implant is defined herein as the midpoint between the poles (18, 19) of space-occupying member. In another embodiment, the size and/or length of the space-occupying member is manually adjusted mechanically. In one embodiment, the space-occupying member is adjustable and is sized/adjusted using a jackscrew or any mechanistic means similar to a jackscrew. In one embodiment, the space-occupying member of the implant device is comprised of more than one segment, wherein the unattached segments are introduced into the abdominal cavity, and the segments are then attached to each other to form an assembled implant device prior to implantation to the outer stomach wall. In one embodiment, the space-occupying member of the implant device comprises a combination of characteristics selected from being compressible, capable of being sized mechanically and comprising attachable segments. In another embodiment, the space-occupying member of the implant device having a preset size does not necessarily preclude the space-occupying member from being made from compressible/flexible material.

In one embodiment of the present invention, a kit is provided that includes more than one implant device, and each implant device of the more than one implant device in the kit has a space-occupying member size different from that of another implant device. As used herein, for the kit, the terms “implant device” and “space-occupying member” are used interchangeably, as the kit would not necessarily include a means for closure. A kit providing more than one sized implant device allows for selection of an appropriate size depending on the anatomy of the patient. A set of dimensions for one space-occupying member includes the length from the pole to pole and the circumference around the midpoint. The depth of the groove or height of the arch can also be included in the set of dimensions. As such, a space-occupying member in the kit will include: space-occupying members having the same length from pole to pole, but varying in circumference around the midpoint; space-occupying members varying in length from pole to pole with the same circumference around the midpoint; and all possible combinations of length and circumference given the ranges for each disclosed herein. Additionally, the space-occupying members having a set of varying dimensions can be provided with a convex arch, a flat “arch”, or have a groove, such that a variety of convexed, flat and grooved (having two arches) implant devices are available in varying lengths and circumferences.

In another embodiment, a kit is provided with multiple implant devices of the same size (having the same set of dimensions), in the ranges disclosed herein. In another embodiment, a kit is provided with multiples of a variety of sizes. For example, a kit may include 2 each of 5 different sizes, for a total of 10 implant devices in the kit. This example is only representative of an example kit, as multiples of a variety of sizes are possible in view of the dimensions and features disclosed herein. As such, a kit as disclosed herein, would have at least two implant devices, and is not otherwise limited to a particular number of implant devices.

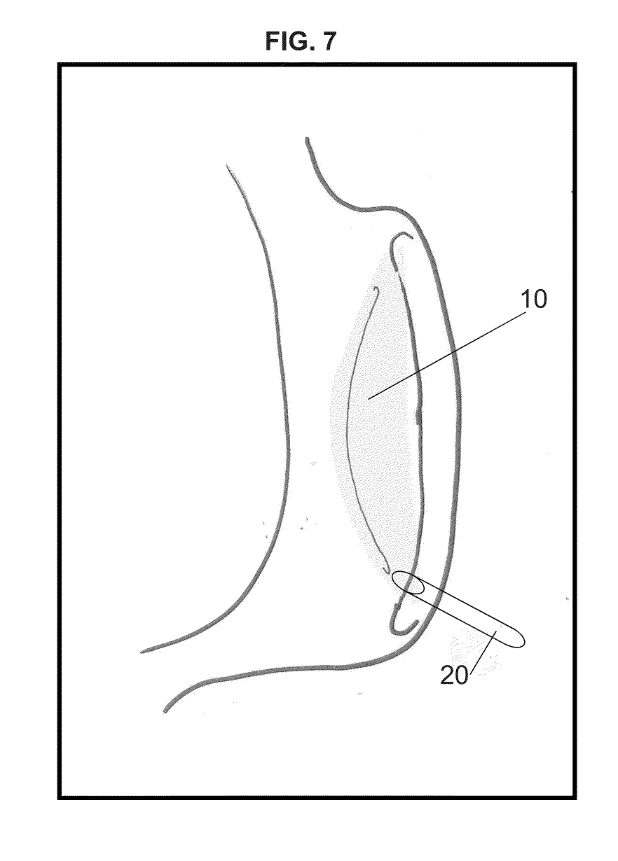

In one embodiment, the space-occupying member of the implant device of the present invention as shown in FIG. 7, has a wand (20) attached to it. The wand (20) is a solid or hollow rod that locks into the space-occupying member of the implant device (10) to allow for manipulation and positioning of the implant. According to the present invention, the wand is used with any surgical implant technique. In one embodiment, a space-occupying member of the implant device (10) having a wand (20) that is introduced into the abdominal cavity by laparoscopic techniques can then be manipulated into position through one of the openings (i.e., trocar sites) by means of the wand (20). Accordingly, the wand has a diameter that is less than the diameter of the opening in the abdominal cavity. In one embodiment, after implantation of an implant device using an attached wand (20), the wand is then released from the space-occupying member (10).

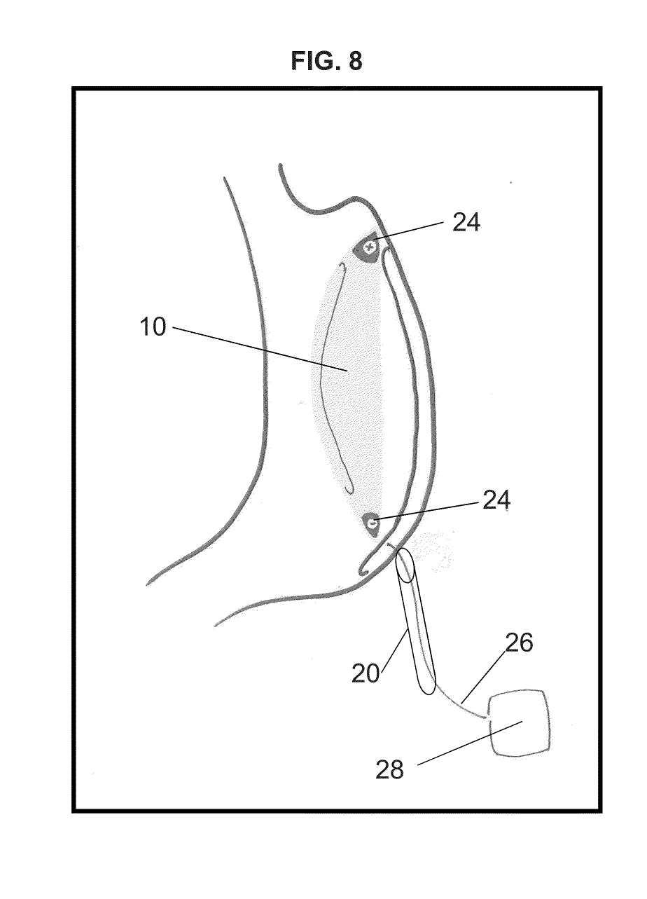

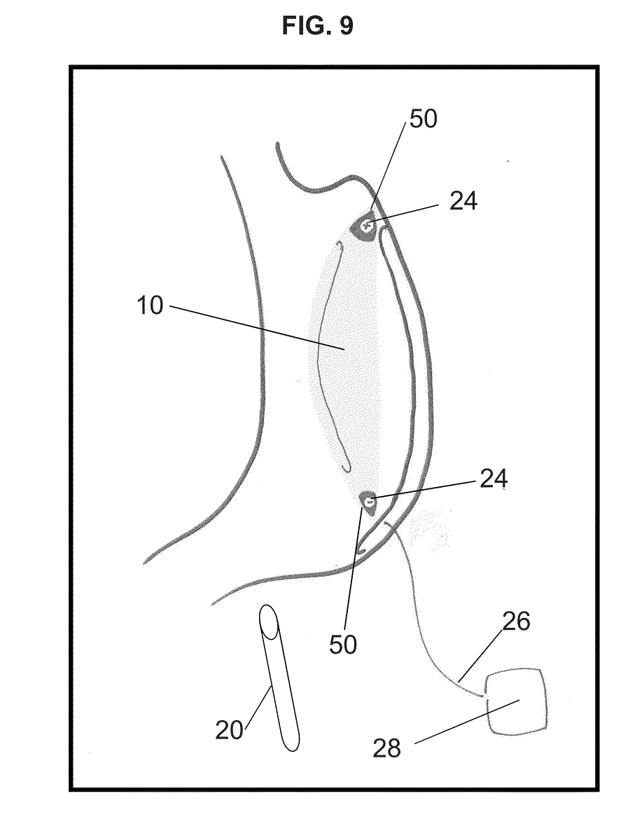

In one embodiment of the present invention, as shown in FIG. 8, the wand (20) attached to the space-occupying member of implant device of the present invention, is hollow and therein encases electrical wire(s) (26) having electrode leads (24) that extend from the implanted space-occupying member (10) at the outer stomach wall to outside the body in order to provide a means to induce myo-gastric function. In one embodiment, (FIG. 9), a space-occupying member (10) is implanted as described herein, electrode leads (24) are placed in the appropriate tissue layer (50), a wand (20) encasing the electrical wire(s) (26) is then released leaving trailing electrical lead wire(s) (26) that can be connected to a power source (28) for gastric pacing or gastric electrical stimulation or inhibition.

In one embodiment, the electrode leads (24) are used for gastric pacing. The electrical leads may have an anode and cathode or other appropriate impulse generating and propagating electrical configuration for the purpose of gastric myo-electrical stimulation/suppression. Gastric pacing (gastric pacemaker) is the use of a set of pacing wires attached to the stomach and an external electrical device that provides a low-frequency, high-energy stimulation to entrain the stomach at a set rhythm (Salvi et al., Ann Ital Chir. 2009; 80(1):25-28). In an alternative embodiment, the implant device of the present invention comprises a pulse generator for inducing gastric electrical stimulation, or any means of high frequency, low energy electrical pulse stimulation (Shikora et al. Surg Obes Relat Dis. 2009; 5(1):31-37).

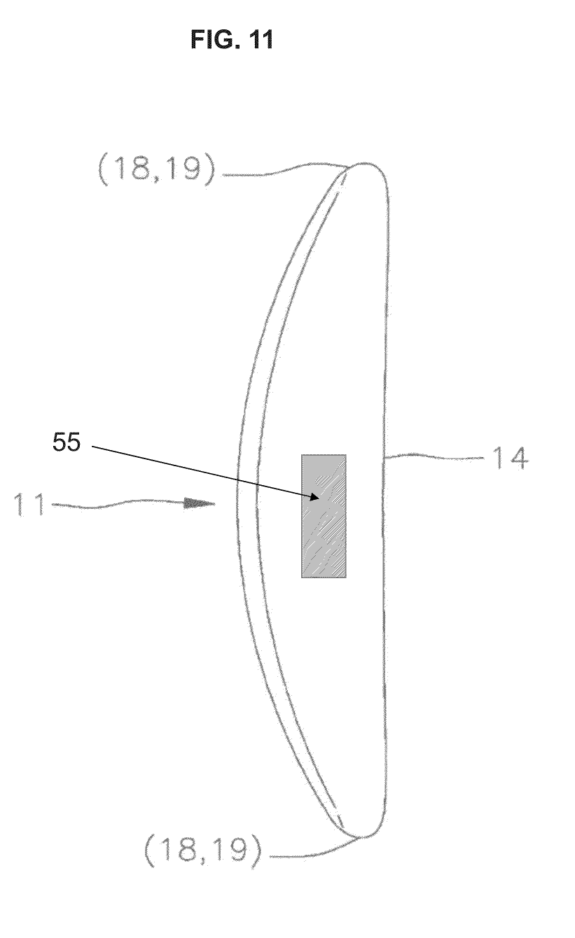

The implant device of the present invention may also contain a computer or processor component (55) (FIG. 11) that allows for wireless interaction with an external source (radio frequency or other) as an energy source or for the unidirectional or bidirectional transmission of signals or data for processes including, but not limited to, geographic location, behavior modification, gastric and/or device pressure or volume evaluation, recording, or management, or physiologic information procurement, maintenance, or management.

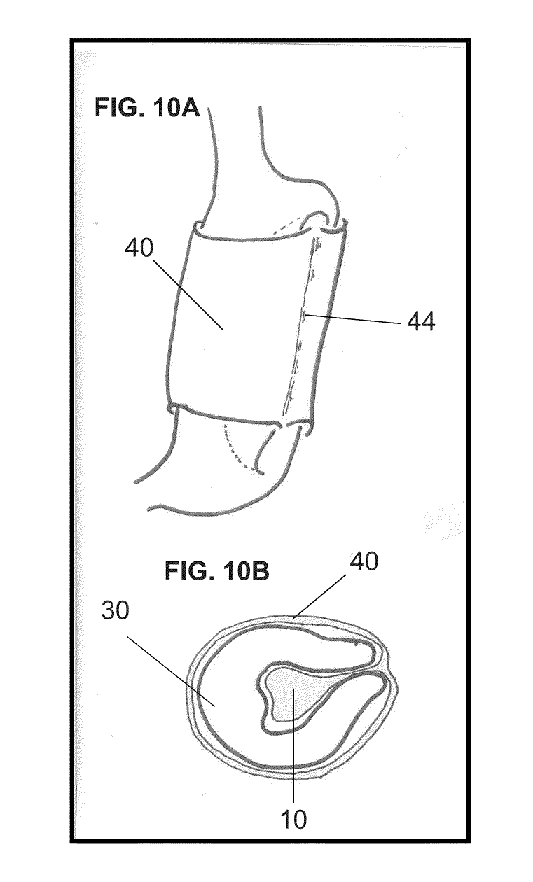

In one embodiment, the space-occupying member of the implant device (10) as disclosed herein, is provided in combination with a wrap device (i.e., extension) (40) (FIGS. 10A-10B). The wrap device (40) is connected to the space-occupying member (10) at the end of imbrication, and extends around the stomach to decrease expansion similar to a corset. This wrap device is positioned around the stomach completely or partially (in a 360-degree or less) fashion and mechanically secured to itself by any mechanical means (44). Examples of means (44) for the wrap device to be secured include sutures, clips, Velcro, any Velcro-like fabric, any biocompatible fabric, as well as any similar means and combinations thereof.

Implantation of the space-occupying member of the implant device (10), according to the present invention, may be accomplished through any applicable surgical procedure. Examples of applicable surgical techniques, include minimally invasive (e.g. laparoscopy), natural orifice (e.g., endoscopy) and/or traditional, “open” surgery. In one embodiment, the implant procedure is performed under general anesthesia. Alternatively, conscious sedation, local anesthesia, or as needed, a combination anesthesia protocol is used. After anesthesia has been provided, the abdominal cavity is entered using any acceptable surgical technique, e.g., open/traditional incision(s), laparoscopy, endoscopy, etc. Laparoscopic surgery can be initiated by accessing the abdominal cavity through a variety of techniques including, but not limited to, needle (e.g., Veress needle) and/or catheter placement, direct trocar/port insertion, or Hasson (“mini-laparotomy”) technique, or combinations thereof (McKernan et al., Endosc Surg Allied Technol., 1995, 3(1): 35-38; Adshead et al., J. Endourol., 2008, 22(2):317-319; Campos et al. J. Laparoendosc Surg., 1991, 1(3): 179-182). The expansion of the abdomen is by any accepted technique, for example, the abdomen is insufflated with gas (e.g., CO₂ or helium), or an abdominal wall lift is employed (The Jap. Assoc of Abd. Wall Lifting for Lap Surg., Surg. Endosc., 1999, 13: 705-709). Various insufflation pressures may be used. As an alternative, or in addition to the open/traditional incision technique or the laparoscopic technique, natural orifice techniques or single incision laparoscopic surgical techniques can be used. Using any of the above incision techniques, anatomical exposure of the stomach is created as needed. For example, the liver is retracted in order to have sufficient access to the stomach (30).

In one embodiment of the present invention, after the stomach is exposed and/or accessed the implant device is placed extra-gastrically using one of the techniques herein, with the division, excision, or surgical manipulation of adjacent tissue as needed to allow the implant device to be placed outside the outer stomach wall. With reference to FIGS. 1 and 2, the greater curve (31), is inverted by the space-occupying member (10) or by another manipulation. Alternatively, the greater curve and/or outer wall of stomach (e.g. the anterior wall) is inverted by the implant device. The space-occupying member is then positioned with the arch (11) oriented toward the lesser curve (32), and the spine oriented to the greater curve (left/lateral side) (31). The greater curve (31) of the stomach is inverted along its length from fundus (36) to antrum (38), and the implant device is placed, or expanded (e.g., by vacuum release or expansion screw or addition of segments) and then placed, proximal to the outer wall (31) of the stomach. If the space-occupying member has an attached wand (20) as described herein, the space-occupying member (10) is positioned into place using the wand. The space-occupying member (10) is then enclosed within the imbricated/inverted stomach, and the enveloping gastric tissue is fixed in apposition. That is, the ends (folds) (33, 35) created by the stomach wall inversion are apposed with any technique that promotes fusion of the external tissue layer through apposition and healing, for example, seromuscular closure. This approach of apposition does not require breaching of the outer stomach wall into the inner stomach. Non-limiting examples of techniques for appositing the stomach ends, include suturing of the ends, wire devices, mechanical pressure devices, stapling devices, shape-memory metals (e.g., nitinol) etc, and combinations thereof.

In one embodiment, a space-occupying member (10) of the present invention, having a wand (20) encasing wires (26) connected to electrode leads (24), is positioned using the wand, and subsequently, the terminal leads are placed in the appropriate tissue layer (50). In another embodiment, the space-occupying member (10) and stomach (30) are then enclosed in the wrap device (40), and the wand (20) is disconnected and withdrawn allowing the wires to trail to the abdominal wall allowing connection to a power source (26). The abdominal cavity is then closed using any accepted technique.

In an alternative embodiment, the space-occupying member (10) is used as a template to create an inversion of the stomach to provide aide for more efficient approximation of the gastric folds/ends (33, 35), and is removed from the body prior to final approximation (closure).

In summary, an implant device and method for inducing weight loss are provided wherein the implant device deforms the stomach wall thereby reducing the effective stomach volume without connection or access to the inner stomach. The implant device of the present invention has a space-occupying member having a shape to provide for deformation of the stomach without retention within the inner stomach.

While the present invention has been described in connection with certain exemplary embodiments, it is to be understood that the invention is not limited to the disclosed embodiments, but, the implant device of the present invention may be of various compositions and be implemented using various techniques within the scope of this invention, without departing from the spirit of the appended claims and equivalents thereof.

What is claimed is:

1. A gastric implant device for decreasing the effective volume of a patient's stomach, comprising:
a solid space-occupying member without a port-type connection, made from biocompatible material adapted to be positioned at an outer stomach wall and to deform the stomach by forming two ends of the outer stomach wall without breaching the inner stomach cavity; and
a means for enclosing the solid space-occupying member by connecting the two ends of the outer stomach wall without breaching the inner stomach cavity.

2. The gastric implant device of claim 1, wherein the solid space-occupying member is homogenous.

3. The gastric implant device of claim 1, wherein the means for enclosing the solid space-occupying member is a seromuscular means selected from the group consisting of sutures, wire devices, mechanical pressure devices, stapling devices, shape-memory metals, and combinations thereof, and wherein the connecting of the two ends of the outer stomach wall is carried out with puncture only to the outer stomach wall.

4. The gastric implant device of claim 1, wherein the biocompatible material is at least one selected from the group consisting of foam, silicone, silastic, polytetrafluoroethylene (PTFE), polyethylene, polypropylene, polyglactin, Seprafilm®, Interceed®, REPEL-CV®, ADEPT®, Biodesign® Surgisis®, human acellular tissue, porcine collagen, bovine pericardium, textured synthetics, and combinations thereof.

5. The gastric implant device of claim 1, wherein the solid space-occupying member further comprises a wand attached thereto.

6. The gastric implant device of claim 5, wherein the wand is hollow and contains wires having electrode leads.

7. The gastric implant device of claim 1, further comprising a wrap for surrounding the stomach.

8. The gastric implant device of claim 1, wherein the solid space-occupying member further comprises a computer or processor.

9. The gastric implant device of claim 1, the solid space-occupying member further comprising:
a first pole and a second pole;
an arch; and
a spine, wherein the arch and spine are positioned opposite each other in between the first pole and the second pole.

10. The gastric implant device of claim 9, wherein the solid space-occupying member further comprises a first arch and a second arch with a groove positioned therebetween, wherein the first and second arches and the groove are positioned opposite the spine.

11. The gastric implant device of claim 9, wherein a length of the solid space-occupying member from the first pole to the second pole is of from about 5 cm to about 25 cm, and a circumference of the solid space-occupying member at a midpoint between the first pole and the second pole is of from about 1 cm to about 15 cm.

12. A method for decreasing the effective volume of a patient's stomach without accessing the inner stomach cavity, comprising:
providing a solid space-occupying member without a port-type connection to an outer stomach wall of the patient;
deforming the stomach with the solid space-occupying member to form two ends of the outer stomach wall;
encasing the solid space-occupying member in the deformed stomach; and
approximating the two ends of the outer stomach wall together to thereby enclose the solid space-occupying member in the outer stomach wall of the deformed stomach.

13. The method of claim 12, further comprising removing the solid space-occupying member prior to approximating the two ends together.

14. The method of claim 12, wherein the solid space-occupying member further comprises a wand and the deforming of the stomach further comprises the step of positioning the solid space-occupying member using the wand.

15. The method of claim 14, further comprising detaching the wand from the solid space-occupying member after approximation of the two ends.

16. The method of claim 15, wherein the wand is hollow and contains wires having electrode leads attached to the solid space-occupying member, further comprising detaching the wand from the solid space-occupying member, wherein the wires having electrode leads remain attached to the solid space-occupying member.

17. The method of claim 16, wherein the electrical leads are used for applying electrical stimulation to the outer stomach wall.

18. The method of claim 12, further comprising surrounding the stomach with an extension wrap.

19. The method of claim 12, wherein the approximating of the two ends is carried out by a seromuscular means selected from suturing, stapling, wiring, mechanical pressure, and combinations thereof.

20. A kit, comprising at least two implant devices of claim 1, wherein each of the at least two implant devices is different in size from that of another implant device of the kit.

21. The kit of claim 20, wherein the solid space-occupying member of each of the at least a first and a second implant devices further comprises:
a first pole and a second pole;
an arch; and
a spine, wherein the arch and spine are positioned opposite each other in between the first pole and the second pole, wherein the solid space occupying member has a set of dimensions comprising a length from the first pole to the second pole that is of from about 5 cm to about 25 cm, and a circumference of the implant device at the midpoint between the first pole and the second pole that is of from about 1 cm to about 15 cm.

22. The kit of claim 21, wherein the solid space-occupying member of at least one of the implant devices further comprises a first arch and a second arch with a groove positioned therebetween, wherein the first and second arches and the groove are positioned opposite the spine.

23. The gastric implant device of claim 1, wherein the solid space-occupying member is compressible.

24. The gastric implant device of claim 1, wherein the solid space-occupying member has an outer material and an inner material.

25. The gastric implant device of claim 24, wherein the inner material is porous.

26. A gastric implant device for decreasing the effective volume of a patient's stomach, comprising:
a solid homogenous space-occupying member made from biocompatible material adapted to be positioned at an outer stomach wall and to deform the stomach by forming two ends of the outer stomach wall without breaching the inner stomach cavity; and
a means for enclosing the solid space-occupying member by connecting the two ends of the outer stomach wall without breaching the inner stomach cavity.
